(19) United States
(12) Patent Application Publication
(10) Pub. No.: US 2026/0033871 A1
(43) Pub. Date: Feb. 5, 2026

(54) SPINOUS PROCESS CLAMP

(71) Applicant: AUGMEDICS LTD., Yokneam (IL)

(72) Inventors: Nissan Elimelech, Beerotaim (IL); Daniel Messinger, Migdal Haemek (IL); Stuart Wolf, Yokneam (IL)

(21) Appl. No.: 19/354,697

(22) Filed: Oct. 9, 2025

Related U.S. Application Data
(63) Continuation of application No. 18/030,072, now Pat. No. 12,458,411.
Application No. 18/030,072 is a continuation-in-part of application No. 17/067,831, filed on Oct. 12, 2020.
Application No. 17/067,831 is a continuation-in-part of application No. 16/120,480, filed on Sep. 4, 2018, now Pat. No. 10,835,296.
(60) Provisional application No. 62/595,598, filed on Dec. 7, 2017.

(51) Int. Cl. A61B 17/70; A61B 17/56; A61B 17/88; A61B 34/20; A61B 90/00

(52) U.S. Cl. CPC A61B 17/7047; A61B 17/7062; A61B 17/8866; A61B 90/39; A61B 2017/564; A61B 17/7056; A61B 34/20; A61B 2034/2055; A61B 2090/365; A61B 2090/376; A61B 2090/3916; A61B 2090/3937; A61B 2090/3966; A61B 2090/3983; A61B 2090/3991

(57) ABSTRACT

Apparatus, consisting of a hinge (84) defining a hinge axis (88), and a pair of opposing jaws (80A, 80B), including a movable jaw and a fixed jaw having a predetermined part radiopaque, so that the fixed jaw location is identifiable from a fluoroscopic image of the fixed jaw. The opposing jaws terminate at proximal and distal regions, and the proximal regions are connected to the hinge so that the movable jaw rotates about the hinge between a closed state and an open state. The jaws are curved in planes parallel to the hinge axis, and terminate in narrowed ends at the distal regions, so that in the closed state the jaws grip sections of vertebrae. The apparatus also has a support structure (60) that retains the hinge and the pair of opposing jaws, and a multiplicity of sharp teeth (98) disposed on respective inner surfaces of the opposing jaws.

### CROSS-REFERENCE TO RELATED APPLICATIONS

This application is a continuation of U.S. patent application Ser. No. 18/030,072, filed Apr. 4, 2023, which is a national stage entry of International Application No. PCT/IB2021/059270, filed Oct. 10, 2021, which is a continuation-in-part of U.S. patent application Ser. No. 17/067,831, filed Oct. 12, 2020, which is a continuation-in-part of U.S. patent application Ser. No. 16/120,480, filed Sep. 4, 2018, which claims the benefit of U.S. Provisional Patent Application No. 62/595,598, filed Dec. 7, 2017. The entire content of each of these related applications is incorporated herein by reference.

### FIELD OF THE INVENTION

The present invention relates generally to clamps, and particularly to clamps that may be attached to the spine of a living subject.

### BACKGROUND OF THE INVENTION

During image guided surgery, it may be important to register elements of a patient, upon whom the surgery is being performed, with equipment generating the image. This is typically the case where the surgery comprises a surgical navigation system, which generates images of portions of the patient that are in registration with the actual portions. Some prior art references that may be used in image guided surgery are provided below.

U.S. Pat. No. 5,665,092 to Mangiardi et al., describes a marker for surgical procedures which permits an operating surgeon to mark the place to be operated on accurately in a manner which is as free of pain as possible for the patient.

U.S. Pat. No. 7,107,091 to Jutras et al., describes a surgical device, adapted for use with an image guided surgical system, that is stated to facilitate monitoring inter-dependently mobile bone elements.

U.S. Pat. No. 8,271,069 to Jascob et al., describes a surgical navigation system for navigating a region of a patient that may include a non-invasive dynamic reference frame and/or fiducial marker, sensor tipped instruments, and isolator circuits. The dynamic reference frame may be placed on the patient in a precise location for guiding the instruments.

U.S. Pat. No. 8,737,708 to Hartmann et al., states that a patient defines a patient space in which an instrument can be tracked and navigated. An image space is defined by image data that can be registered to the patient space. A tracking device can be connected to a member in a known manner that includes imageable portions that generate image points in the image data. Selected image slices or portions can be used to register reconstructed image data to the patient space.

U.S. Pat. No. 8,784,450 to Moskowitz et al., describes thoracic/lumbar and cervical spinous process staples which staple/fuse adjacent spinous processes.

U.S. Pat. No. 9,005,211 to Brundobler et al., describes a method for positioning a guide tube fixation device at a spinal structure of a patient. The method includes attaching an attachment element to the spinal structure, attaching a guide tube to the attachment element, wherein the guide tube is calibrated prior to attachment, and navigating a part of the guide tube to a predetermined location relative to a target region of the patient.

U.S. Pat. No. 9,011,441 to Bertagnoli et al., describes a method for preparing an interspinous space to receive an implantable device.

U.S. Pat. No. 9,060,757 to Lawson et al., describes an instrument for distracting and/or compressing adjacent vertebrae. The instrument includes a yoke, a first blade movably mounted to the yoke, a second blade mounted to the yoke and an adjustment system.

U.S. Pat. No. 9,084,635 to Nuckley et al., describes a number of spinal stabilization devices for aligning and fixing vertebrae during surgery, e.g. to facilitate accurate placement of pedicle screws. One stabilization device includes a pair of spiked rails biased to clamp shut and thereby passively engage a number of vertebrae.

U. S. Patent Application 2015/0282735 to Rossner describes a device and method for a surgical navigation system comprising a connection unit, a marker carrier unit removably attached to the connection unit, and an attachment unit connected to the connection unit for fixing the device to a body part of a patient.

U. S. Patent Application 2016/0022287 to Nehls describes temporary, radiographically opaque, bone markers having first and second penetration members that are concentric with one another and are configured to pierce bone.

U. S. Patent Application 2020/0390502 to Holthuizen et al., describes an arrangement of radiopaque fiducials on a ventricular drain facial mask.

U. S. Patent Application 2020/0129264 to Onativia Bravo et al., describes a reference clamp that can also include radiopaque spheres.

### SUMMARY OF THE INVENTION

An embodiment of the present invention provides apparatus, consisting of: a hinge, defining a hinge axis; a pair of opposing jaws, including a movable jaw and a fixed jaw having a predetermined part thereof radiopaque, so that a location of the fixed jaw is identifiable from a fluoroscopic image of the fixed jaw, the opposing jaws terminating at respective proximal regions and distal regions, wherein the proximal regions are connected to the hinge so that the movable jaw is configured to rotate about the hinge between a closed state and an open state of the jaws, and the jaws are curved in respective planes parallel to the hinge axis, and terminate in respective narrowed ends at the respective distal regions, wherein in the closed state the jaws are configured to grip one or more sections of vertebrae; a support structure configured to retain the hinge and the pair of opposing jaws; and a multiplicity of sharp teeth disposed on respective inner surfaces of the opposing jaws.

In a disclosed embodiment the predetermined part includes a first part of the fixed jaw having a first radiopacity, and a second part of the fixed jaw having a second radiopacity different from the first radiopacity, and the first part is configured so that the fluoroscopic image of the first part provides the location of the fixed jaw. The first part may include a plurality of holes in predetermined locations in the second part. Alternatively or additionally, the first part may include a plurality of beads incorporated into predetermined locations in the second part. Further alternatively or additionally, the first part may include a plurality of the sharp teeth disposed in predetermined locations on an inner surface of the fixed jaw, and the second part may have the fixed jaw absent the first part.

In a further disclosed embodiment the fixed jaw has a single radiopacity and a predetermined shape, and the predetermined shape is configured so that the fluoroscopic image provides the location of the fixed jaw. Typically, the movable jaw has a movable jaw radiopacity different from the single radiopacity.

There is further provided, according to an embodiment of the present invention, a method, consisting of: providing a clamp, including: a hinge, defining a hinge axis; a pair of opposing jaws, including a movable jaw and a fixed jaw having a predetermined part thereof radiopaque, so that a location of the fixed jaw is identifiable from a fluoroscopic image of the fixed jaw, the opposing jaws terminating at respective proximal regions and distal regions, wherein the proximal regions are connected to the hinge so that the movable jaw is configured to rotate about the hinge between a closed state and an open state of the jaws, wherein in the closed state the jaws are configured to grip one or more sections of vertebrae; and using the clamp during an image guided surgery procedure.

In an alternative embodiment, using the clamp during the image guided surgery procedure includes performing a fluoroscopic scan of the clamp in the closed state while the clamp grips the one or more sections of vertebrae, and identifying respective locations of the fixed jaw and the one or more sections of vertebrae from the scan.

The method may also include registering a frame of reference of the fixed jaw with a frame of reference of the one or more sections of vertebrae in response to the identified locations. The method may further include attaching a marker having optical reflectors to the fixed jaw, and tracking a location of the marker in response to an acquired optical image of the marker, so as to track the one or more sections of vertebrae in response to the registered frames of reference.

The present invention will be more fully understood from the following detailed description of the embodiments thereof, taken together with the drawings in which:

### BRIEF DESCRIPTION OF THE DRAWING

FIG. 1 is a schematic illustration of a medical procedure, according to an embodiment of the present invention;

FIG. 2A schematically illustrates the situation after a clamp has been inserted and adjusted in the medical procedure, according to an embodiment of the present invention;

FIG. 2B schematically illustrates how a positioning marker is attached to the clamp, according to an embodiment of the present invention;

FIG. 2C schematically illustrates the positioning marker, according to an embodiment of the present invention; FIGS. 3A, 3B, 3C, 3D and 3E are schematic figures illustrating selected elements of the clamp, according to an embodiment of the present invention;

FIGS. 4A and 4B are schematic figures illustrating further elements of the clamp, according to an embodiment of the present invention;

FIGS. 5A, 5B and 5C are schematic figures illustrating the attachment of the marker to the clamp, according to an embodiment of the present invention.

FIG. 6 is a flowchart of steps performed for the procedure of FIG. 1, according to an embodiment of the present invention;

FIG. 7 is a schematic figure illustrating one of the steps of the flowchart, according to an embodiment of the present invention

FIGS. 8A and 8B are schematic figures illustrating a clamp, according to an alternative embodiment of the present invention;

FIGS. 8C, 8D, and 8E are schematic figures illustrating a clamp, according to a further alternative embodiment of the present invention;

FIG. 9 is a schematic figure illustrating a positioning marker, according to an alternative embodiment of the present invention; and

FIG. 10 is a flowchart of steps performed using a clamp such as the clamp of FIGS. 8A and 8B, or the clamp of FIGS. 8C, 8D, and 8E, and the marker of FIG. 9, according to an embodiment of the present invention.

### DETAILED DESCRIPTION OF EMBODIMENTS

### Overview

Embodiments of the present invention provide apparatus that may be used as a clamp, herein by way of example assumed to be a spinous process clamp, that, in contrast to prior art clamps, has two curved jaws. By curving the jaws, the clamp is able to fasten onto two or more spinous processes of a patient, and yet may be inserted into the patient through a relatively small incision.

A hinge connects the two jaws, one of the jaws being movable so as to rotate about the hinge, the other jaw being fixed with respect to the hinge. The fixed jaw is configured to have a location of the fixed jaw identifiable from a fluoroscopic image of the fixed jaw, by having a predetermined part of the fixed jaw radiopaque.

By configuring a predetermined part of the fixed jaw to be radiopaque, a fluoroscopic scan of the fixed jaw, after the jaws have been fastened on the spinous processes, enables a location of the fixed jaw, and thus the clamp, to be registered with the spinous processes.

The hinge defines a hinge axis, and each jaw is curved in a respective plane parallel to the hinge axis. In addition, each jaw terminates in a respective narrowed region, the narrowed regions facilitating insertion of the jaws of the clamp into the patient via the incision.

Each jaw is configured to have sharp teeth, which enable the clamp, when it is closed, to both cut into and effectively grip the spinous processes.

A clamp support structure retains the two jaws and the hinge. The support structure may be used to manipulate the jaws, after insertion of the jaws into the patient, so that the jaws grip one or more spinous processes of the patient, while the support structure remains external to the patient.

Once the jaws have gripped the spinous processes of the patient, the support structure of the clamp provides a rigid platform to which may be attached a positioning marker that does not move relative to the patient's spine. The clamp with its attached marker may be used during an image guided surgery procedure, in which a professional performing the procedure uses a surgical navigation system.

By virtue of the registration referred to above between the clamp and the spinous processes, the surgical navigation system may find the position and orientation of the marker, and thus of the clamp and the patient, in a frame of reference of the system. Thus any relative movement between the patient and the system, which may be caused by movement of the patient and/or of the professional, may be compensated for.

Using the fixed jaw of the clamp for the registration process enables fluoroscopes with narrow fields of view to perform the scan used for registration. This simplifies the process of registration, in contrast to prior art systems which either require a fluoroscope with a wide field of view, or which require intermediate steps to accommodate the narrow field of view of the fluoroscope, or which require a separate registration marker.

### DETAILED DESCRIPTION

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

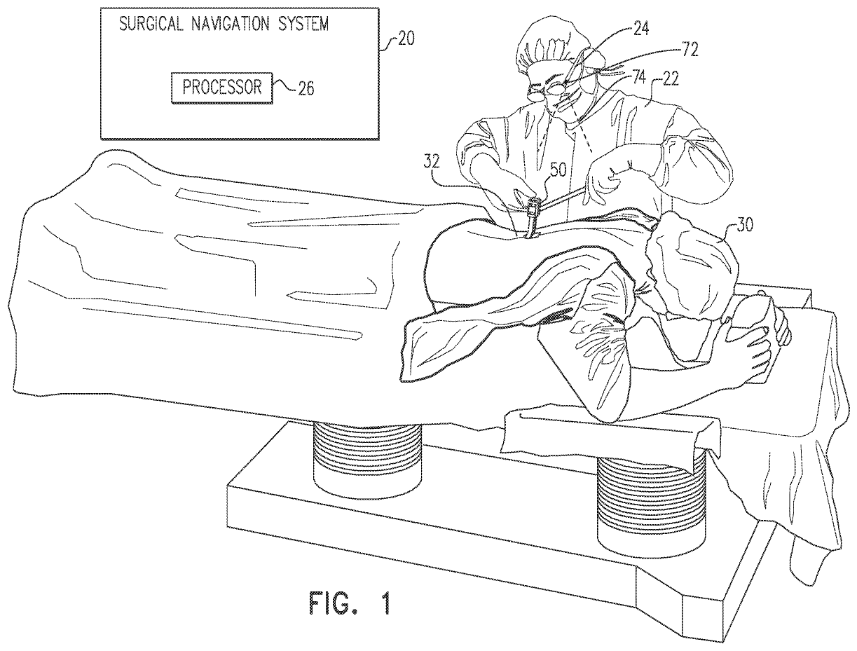

Reference is now made to FIG. 1, which is a schematic illustration of a medical procedure, according to an embodiment of the present invention. During the procedure, performed by a professional 22, the professional uses a surgical navigation system 20, which assists the professional in performance of the procedure. Surgical navigation system 20 comprises a processor 26, which operates elements of the system, and which communicates with an augmented reality assembly 24, worn by professional 22, that is incorporated in the system. Assembly 24 comprises, inter alia, an image capturing device 72, also termed herein a camera 72, that has a field of view 74 and that is configured to capture images in the visible spectrum. Functions of system 20, processor 26, and device 72 are described below. An assembly similar to augmented reality assembly 24, and its operation, are described in U.S. Pat. No. 9,928,629, to Benishti, et al., whose disclosure is incorporated herein by reference.

The medical procedure exemplified here is performed on a patient 30, and during an initial stage of the procedure professional 22 makes an incision 32 into the patient's back. The professional then inserts a spinous process clamp 50 into the incision, so that opposing jaws of the clamp are located on opposite sides of the spinous processes. The professional then slides the clamp over the vertebral laminas, and adjusts the clamp to grip one or more spinous processes, selected by the professional, of the patient. As is described in more detail below, sharp edges of teeth on the jaws of the clamp face forward and are configured to cut muscles to the spinous processes, to facilitate insertion of the clamp. This enables lower sides of the jaws to slide on the vertebral laminas.

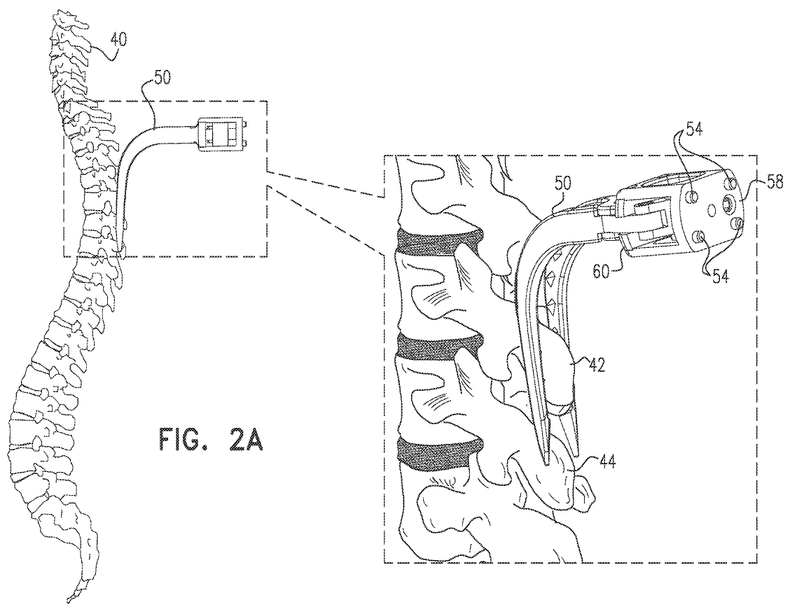

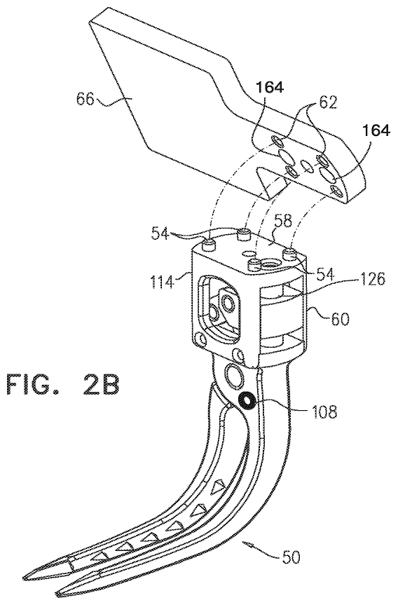

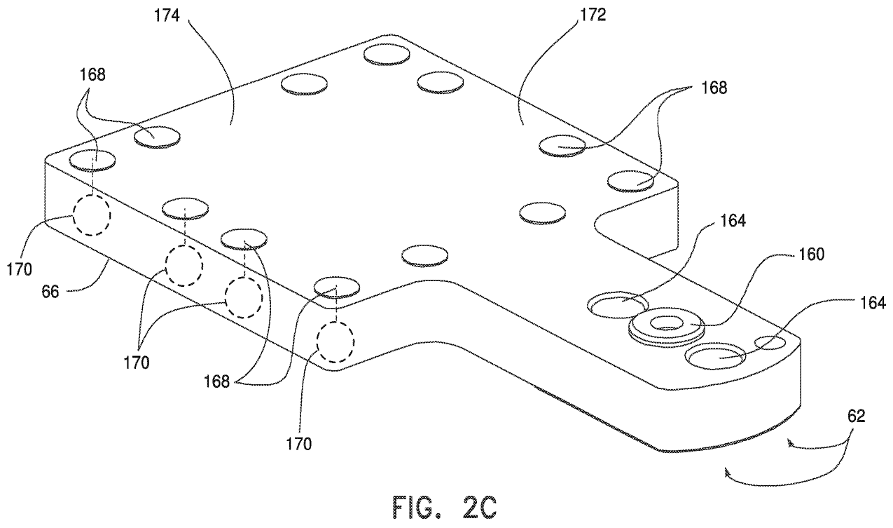

FIG. 2A schematically illustrates the situation after clamp 50 has been inserted and adjusted, according to an embodiment of the present invention. The figure illustrates that clamp 50 has been attached to grip a spine 40 of patient 30, and to specifically grip spinous processes 42 and 44 of vertebrae of the spine. As shown in FIG. 2A, clamp 50 comprises studs 54, herein by way of example there are four studs 54, protruding from a proximal surface 58 of a support structure 60 of the clamp. FIG. 2B schematically illustrates how a positioning marker 66 (shown in more detail in FIG. 2C) is attached to clamp 50, according to an embodiment of the present invention. As is explained below, marker 66 comprises radiopaque and optically visible elements that are in a known alignment with each other. Thus, a fluoroscopic image and an visible spectrum image of the marker may be used to register fluoroscopic and visible spectrum frames of reference of the marker, and of the clamp to which the marker is attached.

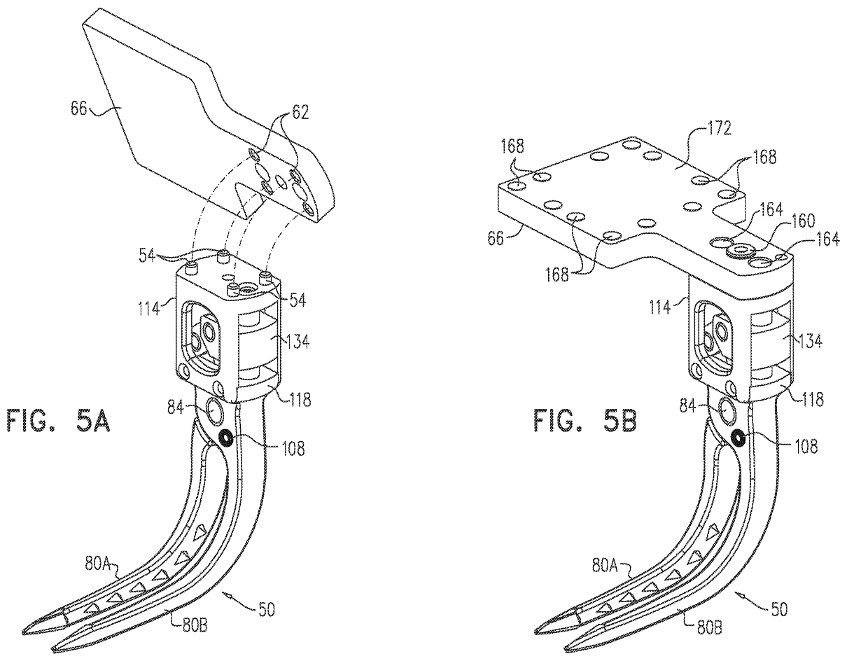

As is illustrated schematically in FIG. 2B, studs 54 of clamp 50 are configured to mate with apertures 62 of marker 66, so that when mated the marker is seated in one of a multiplicity of selectable positions on surface 58. In the embodiment described herein there are two selectable positions, and FIG. 2B illustrates the marker attached in one of those positions—to the left of the clamp (FIG. 5C shows the marker attached to the right of the clamp). However, it will be understood that the studs and apertures may be configured so that there are more than two selectable positions in which marker 66 may be seated on surface 58.

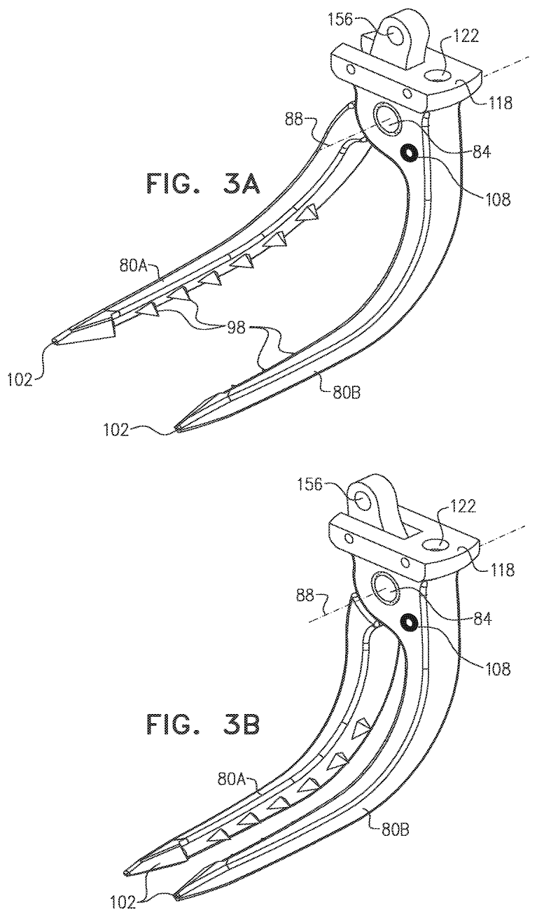

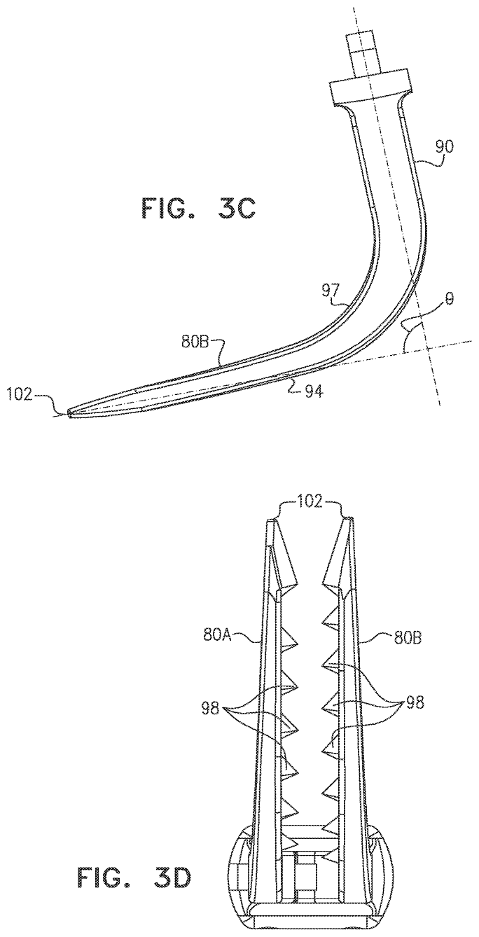

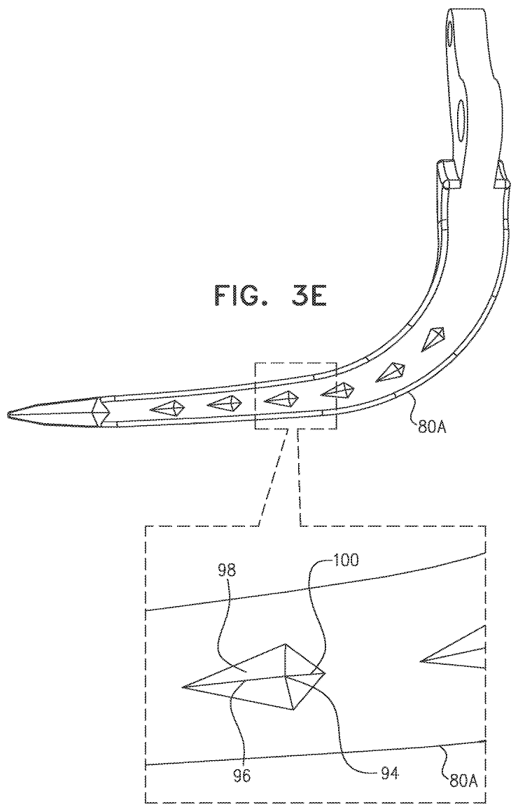

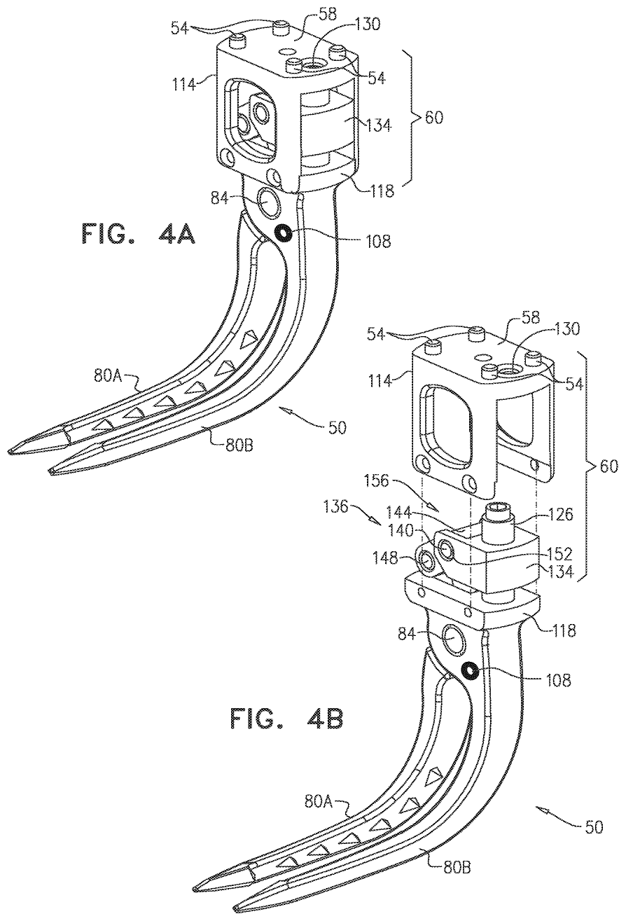

FIG. 2C schematically illustrates details of marker 66, according to an embodiment of the present invention. Marker 66 is formed of a generally rectangular sold substrate 174, which comprises access holes 164 to a set screw 126 (described below with reference to FIG. 4B), and the holes are configured so that, regardless of the direction of attachment of the marker to the clamp, there is access to the set screw, via one of holes 164, after the marker has been attached.

During the procedure performed by professional 22, marker 66 is used as a fiduciary facilitating tracking of, and compensating for, any apparent movement of clamp 50 relative to assembly 24. The marker is also used to enable registration of a visible spectrum frame of reference of the marker with a CT frame of reference of the marker.

In order to act as a fiduciary, marker 66 comprises a multiplicity of reflectors 168 that are arranged on an upper face 172 of substrate 174 in a predetermined pattern. Reflectors 168 reflect visible light, and substrate 174 is typically opaque in the visible spectrum. When the reflectors are illuminated, by a projector (not shown) of surgical navigation system 20 and/or by ambient visible light, image capturing device 72 forms an image of the reflectors, and the image is transferred to processor 26. To act as a registration device, marker 66 also comprises a multiplicity of radiopaque elements 170 that are arranged in a known predetermined physical relationship with respect to reflectors 168, and that are typically embedded in substrate 174. By way of example, elements 170 are assumed to be a preset distance directly below reflectors 168, and FIG. 2C shows four such elements as spheres. The material of substrate 174 is selected to be transparent under fluoroscopy.

In some embodiments reflectors 168 and radiopaque elements 170, rather than comprising separate entities as describe above, are comprised of combined single elements that are both reflective in visible light and radiopaque. For example, reflectors 168 may be formed from aluminum discs.

FIGS. 3A, 3B, 3C, 3D and 3E are schematic figures illustrating selected elements of clamp 50, according to an embodiment of the present invention. Clamp 50 comprises two opposing jaws 80A, 80B, which are connected, at proximal regions of the jaws, by a hinge pin 84. Jaws 80A, 80B are also herein termed jaws 80. At least one of the jaws is able to rotate around hinge pin 84, and in the figures jaw 80A is shown as rotating about the hinge pin, while jaw 80B is fixed with respect to the pin. The rotation enables jaws 80 to transfer in a continuous manner between an open state of the jaws, illustrated in FIG. 3A, and a closed state of the jaws, illustrated in FIG. 3B.

Hinge pin 84 defines a hinge axis 88, and each jaw 80 resides in a respective plane parallel to the hinge axis. FIG. 3C illustrates jaw 80B, and the figure has been drawn so that the plane of the paper of FIG. 3C corresponds to the plane wherein jaw 80B resides.

Each jaw 80 is curved within its respective plane. Thus, as illustrated in FIG. 3C, jaw 80B comprises a first substantially straight proximal region 90, a second substantially straight distal region 94, and a curved intermediate section 97 fixedly connecting the two straight regions. Proximal region 90 and distal region 94 are both terminating regions of the jaw 80B. Jaw 80A comprises two similar regions and a similar intermediate section that are connected as for jaw 80B. Typically, the distal regions of the jaws are sufficiently long so that they are able to simultaneously clamp on multiple adjacent spinous processes. In a disclosed embodiment the jaws are approximately 7 cm long. In one embodiment an angle θ made by the intersection of the two straight sections is approximately 90°. However, in other embodiments, angle θ may be in the approximate range of 70°-90°.

Typically each jaw 80 has a multiplicity of teeth 98 on an inner surface of the jaw, and the teeth of the two jaws are configured to be in opposition to each other. In one embodiment the opposing teeth are configured to intermesh with each other, as is illustrated in FIG. 3D, when the jaws are in their closed state.

In a disclosed embodiment the teeth have a pyramid-like shape, as illustrated in FIG. 3E, wherein the edges of the pyramid are sharp. In this case the pyramid of each tooth is configured to terminate in a sharp point 94, which facilitates the tooth gripping a spinous process.

In addition, for each tooth 98 one edge 96 of the pyramid is configured to be sharp and to face forward, so as to enable cutting of the muscles between the spinous processes when the clamp is inserted. Furthermore, another edge 100 of the pyramid of each tooth 98 is also configured to be sharp, but to face backward, so as to enable cutting of the muscles between the spinous processes on removal of the clamp. It will be understood that the forward-facing sharp edges of the pyramid-shaped teeth facilitate insertion of the clamp into patient 30, and that the backward-facing sharp edges of the pyramid-shaped teeth facilitate removal of the clamp from the patient.

Distal terminations 102 of each distal section of jaws 80A and 80B are narrowed by being slightly truncated and rounded, so that the distal tips of the jaws are not sharp. This configuration facilitates insertion of the clamp into soft body tissue, such as muscle or fat. In an alternative embodiment, edges 104 of internal faces 106 of terminations 102 are sharp so as also to facilitate penetration through tissue during insertion of the jaws.

In one embodiment jaws 80 are formed of anodized aluminum, typically black anodized aluminum for biocompatibility. In other embodiments jaws 80 may be formed of other metallic or non-metallic materials, including composites, having substantially the same physical properties as aluminum.

The proximal region of jaw 80B comprises a support structure retaining base 118 (FIGS. 3A, 3B) that is fixed to jaw 80B and which in turn comprises a blind aperture 122. The functions of base 118 and aperture 122 are described below. Clamp 50 also comprises a verification point 108 which is located in a known, predefined, fixed position on the clamp, and is herein assumed to be fixed, by way of example, to non-movable jaw 80B. However, verification point 108 may be located on other fixed portions of clamp 50, such as support structure 60.

Point 108 is situated on clamp 50 so that after the clamp has been inserted into patient 30, the verification point is visible to professional 22, when illuminated by visible light. In addition to being visible to professional 22, the verification point is configured to be identifiable, typically by image segmentation, in a fluoroscopic image, such as a computerized tomography (CT) image of clamp 50.

In order to be identifiable as described above, verification point 108 may comprise a protuberance and/or an indentation in jaw 80B, the protuberance and/or indentation being in the form of any convenient shape such as a cylinder. Alternatively or additionally, verification point 108 may be comprised of a material that is different from the material of clamp 50 wherein the point is located, so long as the material of the point is distinguishable in a fluoroscopic image, and also when viewed in visible light, from the material of the clamp. For example, if jaw 80B is formed from aluminum, point 108 may be formed from a titanium element, such as a spherical bead, that is inserted into the jaw, and that protrudes from the jaw. As another example, point 108 may comprise a void, such as a spherical air pocket, in jaw 80B, and the position of the point may be made visible by an optically visible marking, such as a paint spot, on a surface of the jaw above the void.

Other methods for forming verification point 108, including but not limited to combinations and sub-combinations of those described herein, will be apparent to those having skill in the art, and all such methods are assumed to be comprised within the scope of the present invention.

Verification point 108 is used by professional 22 to verify that different elements of system 20 are in registration, as is described in more detail below.

FIGS. 4A and 4B are schematic figures illustrating further elements of clamp 50, according to an embodiment of the present invention. FIG. 4A illustrates the clamp when assembled, and shows support structure 60 which retains and activates jaws 80 and hinge 84. FIG. 4B illustrates support structure 60 with a cover 114 of the structure translated from other elements of the structure. Cover 114 is fixedly attached to support structure retaining base 118 (FIGS. 3A, 3B)

Support structure 60 contains a set screw 126 which is retained by, and rotates within, blind aperture 122 and an open aperture 130 in cover 114. Screw 126 has an external thread which mates with an internal thread of a nut 134, and sides of the nut touch and slide within walls of cover 114. Thus, rotating screw 126 translates nut 134 vertically within cover 114.

Nut 134 is connected by a lever mechanism 136 to jaw 80A. Mechanism 136 comprises a first hinge pin 140, a lever rod 144, and a second hinge pin 148. Rod 144 connects, at its proximal end, via first hinge pin 140, to an aperture 152 in nut 134, so that the rod is able to rotate around the first hinge pin. Rod 144 connects, at its distal end, via second hinge pin 148 to an aperture 156 in jaw 80A (aperture 156 is also shown in FIGS. 3A, 3B.)

Lever mechanism 136 is operated by rotating set screw 126. When the screw is rotated so that nut 134 has translated in an upper direction within structure 60, hinge 148 and aperture 156 are drawn towards the set screw, so that jaws 80 are in their open state, illustrated in FIG. 3A. When set screw 126 is rotated so that nut 134 has translated in a lower direction within structure 60, hinge 148 and aperture 156 move away from the set screw, so that jaws 80 are in their closed state, illustrated in FIG. 3B.

It will be understood that set screw 126, as it acts on lever mechanism 136, fixedly maintains jaws 80 in any desired configuration according to the rotation of the set screw. Thus a first rotation of screw 126 can fixedly maintain the jaws in an open state, and a second rotation can fixedly maintain the jaws in a closed state.

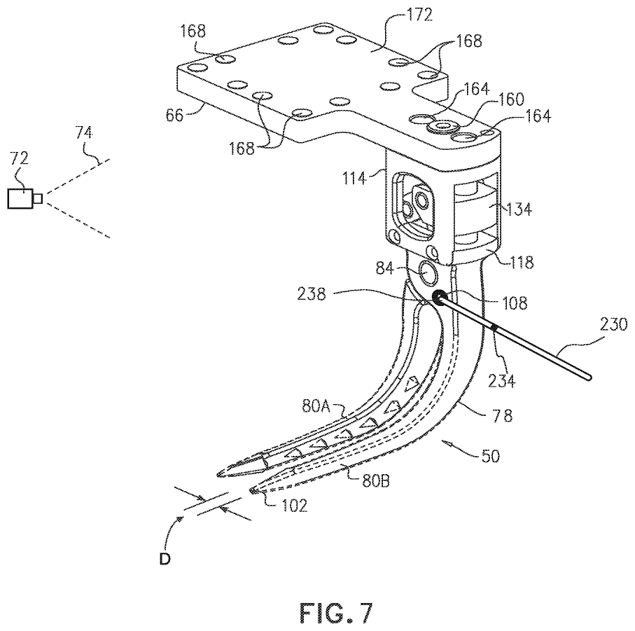

During the procedure referred to above, in their closed state the jaws typically grip one or more spinous processes. In order to firmly grip the processes in their closed state, in an embodiment the jaws are configured, by selection of the material of the jaws and by selection of the jaw dimensions, to bend so that respective distal terminations 102 of the jaws deflect by up to 1 mm from the jaw configuration when they are not gripping the processes. The deflection caused by the bending when the jaws grip spinous processes is illustrated schematically in FIG. 7.

From the descriptions of clamp 50 above, it will be appreciated that the combination of the narrowed distal sections of the jaws, their curved shape, and the relatively long length of the distal straight sections means that even with a small incision 32, clamp 50 can be manipulated to effectively grasp multiple spinous processes of patient 30.

FIGS. 5A, 5B and 5C are schematic figures illustrating the attachment of marker 66 to clamp 50, according to an embodiment of the present invention. FIG. 5A illustrates apertures 62 of the marker mating with studs 54 of the clamp, and FIGS. 5B and 5C illustrate the marker and the clamp when they are attached. In the embodiment illustrated, marker 66 may be attached to clamp 50 so that the marker is to the left of the clamp, as shown in FIG. 5B, or to the right of the clamp, as shown in FIG. 5C. The marker is attached to the clamp with a screw 160.

During a procedure professional 22 can select which direction marker 66 is in so as to have best access to the patient. Typically, after insertion of jaws 80 into the patient, support structure 60 is approximately perpendicular to the patient's spine.

As is described above, marker 66 comprises access holes 164 to set screw 126, and the holes are configured so that, regardless of the direction of attachment of the marker to the clamp, there is access to the set screw, via one of holes 164, after the marker has been attached.

As is also described above, professional 22 may use surgical navigation system 20 (FIG. 1) during the procedure being performed on patient 30, and marker 66 may be used by the system as a fiduciary, enabling any relative movement between the patient and the system to be compensated for. In order to operate as a fiduciary, processor 26 of the surgical navigation system may use image capturing device 72 to recognize an image of the marker itself, or of optical elements such as reflectors 168 of the marker.

Thus, when reflectors 168 are illuminated by a projector (not shown) of surgical navigation system 20, and/or are illuminated by ambient visible light, image capturing device 72 forms an image of the reflectors, and the image is transferred to processor 26. Processor 26 uses the captured image to find the position and orientation of marker 66 and of clamp 50, and thus the position and orientation of the patient's spine, onto which clamp 50 is clamped, in a frame of reference defined by the system. Consequently, processor 26 is able to compensate for any relative movement between the marker and the system, which may be caused by movement of patient 30 and/or of professional 22, and the compensation enables the processor to adjust images presented to professional 22 so that the adjusted images appear stable with respect to the patient's spine.

Other methods for using marker 66, to compensate for any movement of patient 30, will be apparent to those having ordinary skill in the art, and all such methods are assumed to be comprised within the scope of the present invention.

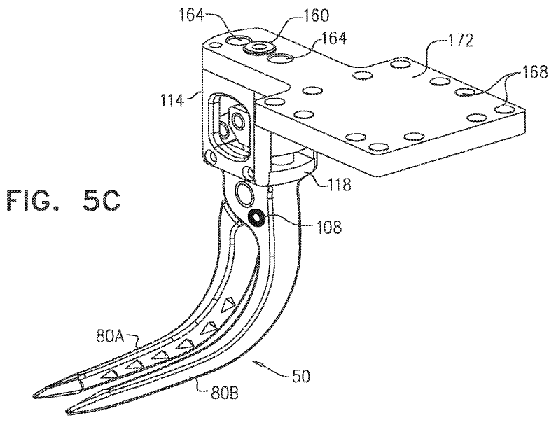

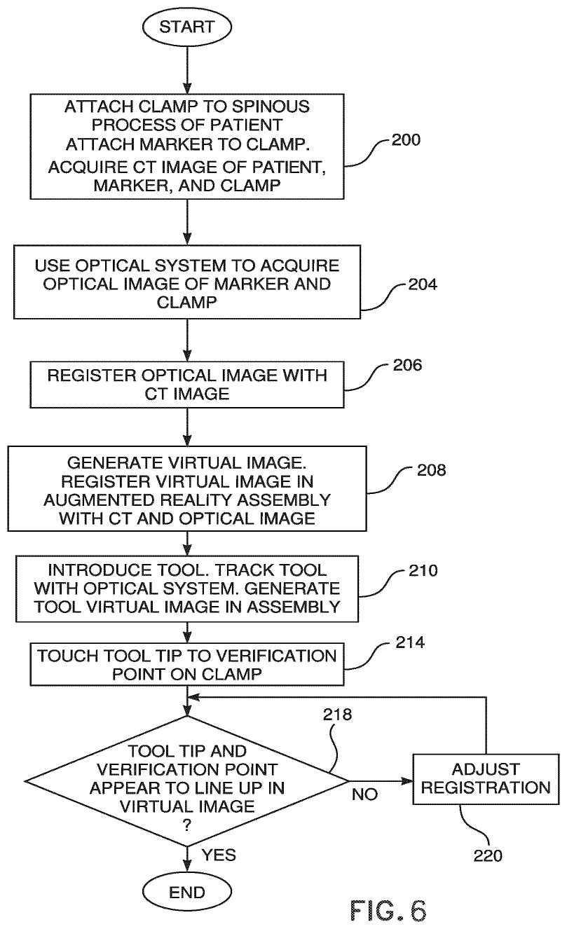

FIG. 6 is a flowchart of steps performed by professional 22 and processor 26 in operation of system 20, and FIG. 7 is a schematic figure illustrating one of the steps, according to an embodiment of the present invention. In an initial step 200 clamp 50 is inserted into patient 30 and is attached to one or more spinous processes of the patient, as is described above. Marker 66 is attached to clamp 50, as described above with reference to FIGS. 5A, 5B, 5C.

Once the clamp and marker are attached, a CT image of the patient, the clamp, and the marker is acquired, and processor 26 stores the image. The CT image includes a fluoroscopic image of verification point 108.

In an optical imaging step 204 the professional activates camera 72 to acquire an optical image of the marker, and processor 26 stores the optical image.

In a registration step 206, processor 26 registers a frame of reference defined by the CT image of marker 66 acquired in step 200 and a frame of reference defined by the optical image of marker 66 acquired in step 204 by methods which are well known in the art. It will be understood that the registration can be performed because reflectors 168, visible in the optical image, and radiopaque elements 170, visible in the CT image, are in a known physical relationship with respect to each other.

In a virtual image step 208, processor 26, using camera 72, generates a virtual image of the scene viewed by professional 22, herein assumed to comprise clamp 50, marker 66, and the spine of patient 30. The processor registers a frame of reference of the virtual image with the registered CT and optical frames of reference, and projects the registered virtual image in augmented reality assembly 24 so that it is visible to the professional.

The registrations produced in steps 206 and/or 208 may be inaccurate, and the following steps of the flowchart allow professional 22 to check on the accuracy of the registrations, and if necessary to correct the registrations. FIG. 7 schematically illustrates clamp 50 inserted into patient 30, and a lower part 78 of the clamp that is within the patient is shown as drawn with broken lines. For simplicity the spinous processes of the patient are not shown in the figure. A deflection D, typically approximately 1 mm, of termination 102 of jaw 80B, caused by bending of the distal region of the jaw when it grips the spinous processes, is illustrated schematically in the figure. Jaw 80A will undergo a similar bending and termination deflection.

FIG. 7 also schematically illustrates camera 72 and field of view 74. It will be understood that the image formed by camera 72 includes images of the upper part of clamp 50 and marker 66, but does not include an image of lower part 78.

In a tool presentation step 210, professional 22 introduces a tool 230 (FIG. 7) into field of view 74 of camera 72. Tool 230 has one or more optical tracking elements 234 located in predefined positions on the tool, and the elements are configured so that an image of tool 230 and elements 234 produced by camera 72 enable processor 26 to track the position of a distal tip 238 of the tool.

The processor generates a virtual image of tool 230, and projects the tool virtual image in augmented reality assembly 24 so that it is visible to professional 26.

In a verification step 214, professional 26 touches distal tip 238 on verification point 108. This step may be performed with the virtual image produced by assembly 24 switched on or switched off.

In a decision step 218, the professional observes if the images of distal tip 238 and verification point 108 coincide. In an embodiment, the position of verification point 108, which is determined from the CT image acquired in step 200, is incorporated in the registered image being presented to the professional by assembly 24. Alternatively, rather than the professional checking for coincidence, processor 26 checks for coincidence.

If decision step 218 returns positive, i.e., the two images coincide, the registrations of the system described above are assumed successful, and the flowchart ends.

If decision step 218 returns negative, the registrations of steps 206 and/or 208 are not sufficiently correct, and control continues first to an adjust registration step 220, and from there to decision step 218. In adjust registration step 220 the processor uses the image generated by camera 72 to determine 3D coordinate values, in the optical frame of reference of marker 66, of distal tip 238 and verification point 108. There is a difference in these coordinate values, illustrated by the negative return of decision step 218, corresponding to a gap between the two values. The processor then adjusts the registrations performed in steps 206 and 208 in order to reduce the gap and returns, iteratively, to decision step 218. The iterations of decision step 218 and adjustment step 220 continue until decision step 218 returns positive.

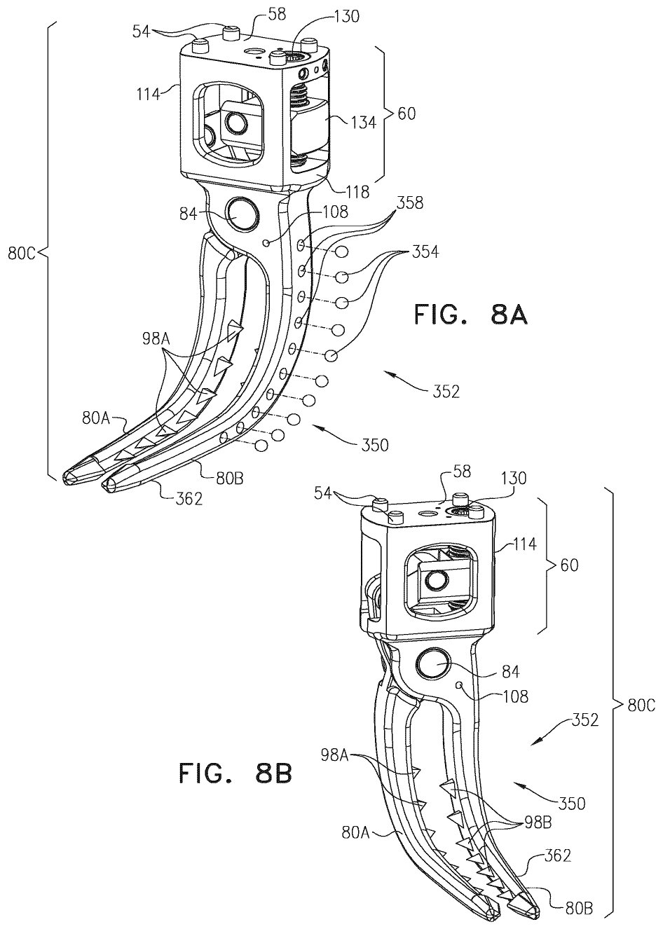

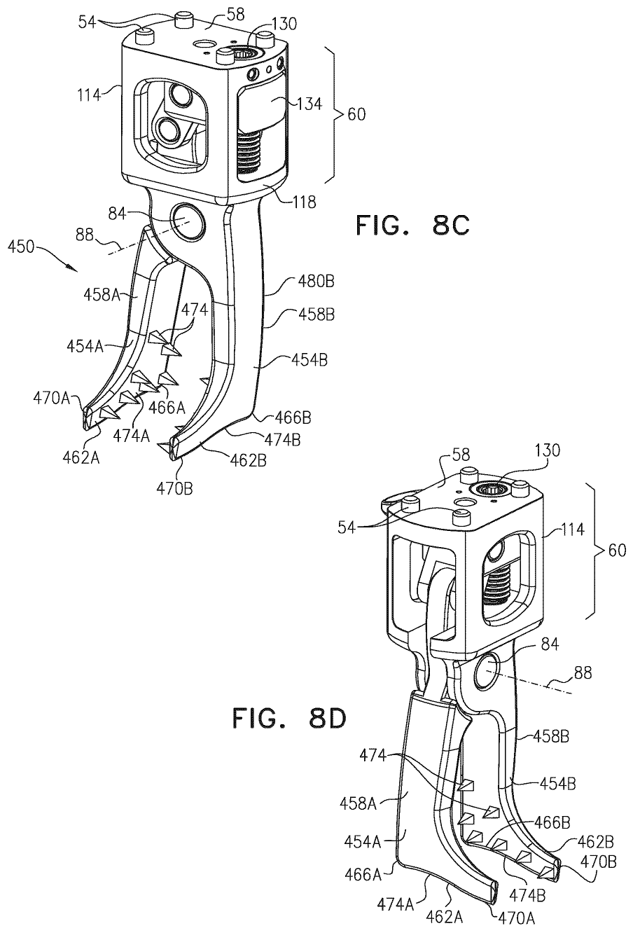

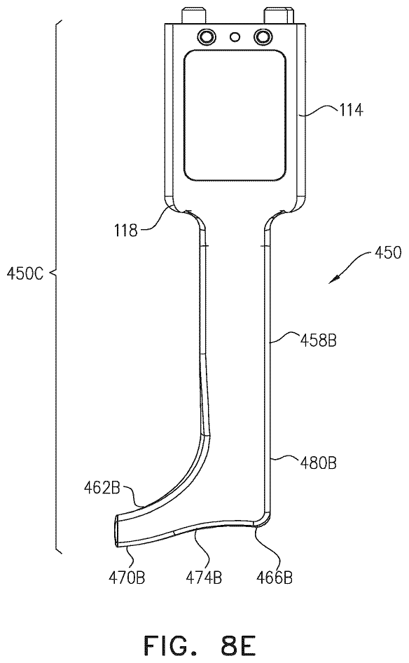

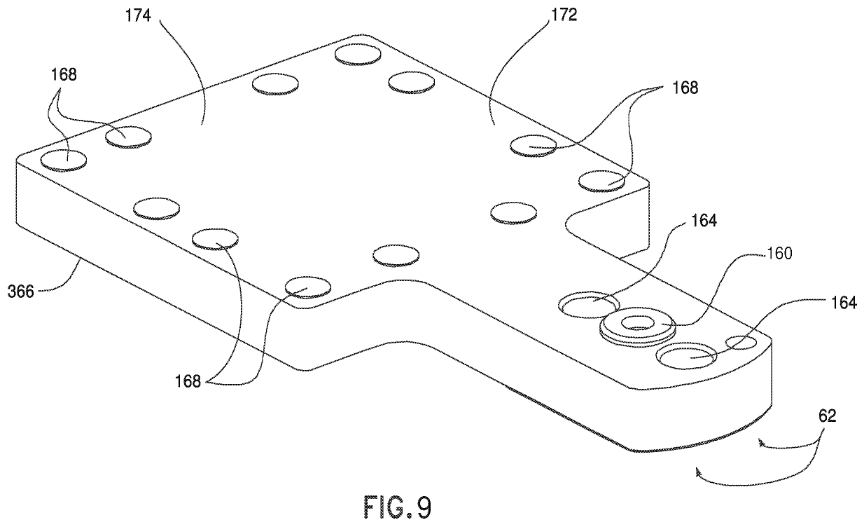

FIGS. 8A and 8B are schematic figures illustrating a clamp 350, according to an alternative embodiment of the present invention, and FIGS. 8C, 8D, and 8E are schematic figures illustrating a clamp 450, according to a further alternative embodiment of the present invention. FIG. 9 is a schematic figure illustrating a positioning marker 366 which is used with clamp 350 or clamp 450, according to an alternative embodiment of the present invention. Apart from the differences described below, the operations of clamps 350 and 450, and of marker 366, are generally similar to that of clamp 50 and marker 66, and elements indicated by the same reference numerals in the clamps and the markers are generally similar in construction and in operation.

Referring to FIGS. 8A and 8B, in contrast to clamp 50, which has cover 114 of support structure 60 separate from, but fixedly connected to, support structure retaining base 118 and jaw 80B, in clamp 350 jaw 80B, cover 114, and base 118 are formed as one single piece, herein termed jaw assemblage 80C. During operation of clamp 350, jaw 80A rotates about hinge 84, whereas jaw 80B, comprised in jaw assemblage 80C, is fixed with respect to the hinge. Jaw assemblage 80C is herein also referred to as fixed jaw assemblage 80C, and jaw 80B is herein also referred to as fixed jaw 80B.

In a disclosed embodiment of the present invention, jaw 80B is configured so that a selected part 352 of the jaw has a different radiopacity from remaining parts of the jaw. When selected part 352 is in a predetermined spatial relationship in jaw 80B, the difference in radiopacity of the selected part is such that in a fluoroscopic image of jaw 80B the image of the selected part is distinguishable from the image of the remaining parts, so that a location of jaw 80B may be deduced from the fluoroscopic image of selected part 352 of the jaw. Selected part 352 typically comprises a plurality of elements incorporated into jaw 80B. The number, locations, and shapes of the elements of part 352 are chosen so that a fluoroscopic image of the elements enables deduction of the location of jaw 80B.

As a first example of a disclosed embodiment, selected part 352 comprises a plurality of blind cylindrical holes 358, formed in predetermined locations in jaw 80B. In this first example, holes 358 are open to the atmosphere, and so are air-filled. The remaining part of jaw 80B and of assemblage 80C may be formed from any suitable material, such as aluminum or other metallic or non-metallic materials, including composites having generally the same physical properties as aluminum. In a disclosed embodiment the inventors use seven holes 358, but embodiments of the present invention may use more or fewer numbers of holes. It will be understood that the radiopacity of the remaining part of jaw 80B and of the remainder of assemblage 80C is different from the radiopacity of selected part 352, i.e., holes 358.

As a second example of a disclosed embodiment, selected part 352 comprises a plurality of beads 354 cemented into holes 358, so that the beads are in the predetermined locations of the holes. FIG. 8A is an exploded illustration showing beads 354 removed from holes 358. The material of beads 354, i.e., selected part 352, is chosen so that the radiopacity of the beads is different from the radiopacity of the remaining part of jaw 80B and of assemblage 80C. In a disclosed embodiment the inventors use seven beads 354, but embodiments of the present invention may use more or fewer numbers of beads. In a disclosed embodiment beads 354 are formed from titanium alloy, and the remaining part of jaw 80B and the remainder of assemblage 80C are formed from a material having a radiopacity different than titanium, such as aluminum. In one embodiment beads 354 are formed as spherical balls, but it will be understood that beads 354 may comprise any convenient shape.

As a third example of a disclosed embodiment, selected part 352 comprises a plurality of teeth 98B that are formed in predetermined locations on an internal surface of jaw 80B, and teeth 98B are comprised in teeth 98. The material of the plurality of teeth 98B, i.e., selected part 352, is chosen so that the radiopacity of the teeth is different from the radiopacity of the remaining part of jaw 80B and of the remainder of jaw assemblage 80C. Typically the material of the remaining part of jaw 80B and of the remainder of jaw assemblage 80C is selected to be the same material as that of jaw 80A. Jaw 80A comprises teeth 98A that are formed on an internal surface of jaw 80A, and teeth 98A are comprised in teeth 98.

In the third example, by having jaw 80A, including its teeth 98A, the remaining part of jaw 80B, and the remainder of assemblage 80C formed from the same material, then in a fluoroscopic image of clamp 350, images of teeth 98B are distinguishable from the image of the remainder of the clamp. Consequently, the location of jaw 80B may be deduced from the fluoroscopic image of the clamp. In a disclosed embodiment teeth 98B are formed from titanium alloy, and the other sections of clamp 350 referred to above are formed from a material having a radiopacity different than titanium, such as aluminum.

Other examples of jaw 80B, comprising a selected part of the jaw having a radiopacity different from the radiopacity of the remaining part, including combinations of the examples given above, will be apparent to those having ordinary skill in the art, and all such other examples are assumed to be comprised within the scope of the present invention.

In an alternative embodiment of the present invention, jaw 80B and jaw assemblage 80C is formed of one or more materials with a common radiopacity, so that in the alternative embodiment there is no part 352 with a different radiopacity. In the alternative embodiment, the one or more materials are selected to have a radiopacity different from elements in proximity to clamp 350 when the clamp is used in a surgical procedure. For example, if clamp 350 is used to clamp spinal processes, then the one or more materials of the alternative embodiment have a common radiopacity that is different from the radiopacity of the spinal processes.

In the alternative embodiment, jaw 80B is formed in a predetermined three-dimensional (3D) shape 362, and the shape is configured so that a location of the jaw can be determined from a fluoroscopic image of the clamp. In one embodiment jaw 80B is formed from aluminum.

Referring to FIGS. 8C, 8D, and 8E, FIGS. 8C and 8D are perspective views of clamp 450 and FIG. 8E is a side view of a portion of clamp 450. As for clamps 50 and 350, clamp 450 comprises two opposing jaws 454A and 454B which are connected, at proximal regions of the jaws, by hinge pin 84. Jaws 454A and 454B are herein also termed jaws 454. As for jaws 80 of clamps 50 and 350, jaws 454A and 454B of clamp 450 also comprise respective substantially straight proximal regions 458A and 458B, herein also termed regions 458. However, in contrast to jaws 80, jaws 454 do not comprise a straight distal region, and also do not comprise a curved intermediate section. Rather, jaws 454A and 454B comprise respective distal “foot-like” regions 462A and 462B, herein also termed feet 462, the regions having an outline in the shape of a human foot. Thus foot 462A comprises a heel section 466A, a toe section 470A, and an arch section 474A connecting the heel and toe sections. Similarly foot 462B comprises a heel section 466B, a toe section 470B, and an arch section 474B connecting heel section 466B with toe section 470B. The toe sections are narrowed regions of the foot-like regions.

Jaw 454A is formed by heel section 466A being connected directly and approximately orthogonally to a distal termination of straight region 458A, so that foot 462A and straight region 458A reside in a common plane and form one single piece. Similarly jaw 454B is formed by heel section 466B being connected directly and approximately orthogonally to a distal termination of straight region 458B, so that foot 462B and straight region 458B reside in a common plane and, together with other elements described below, form one single piece. Each jaw 454 resides in a respective plane parallel to hinge axis 88. FIG. 8E illustrates, inter alia, jaw 454B, and the figure has been drawn so that the plane of the paper of FIG. 8E corresponds to the plane wherein jaw 454B resides.

As for clamp 350, and as is illustrated in FIG. 8E, in clamp 450 jaw 454B, cover 114, and base 118 are formed as one single piece, herein termed jaw assemblage 454C. During operation of clamp 450, jaw 454A rotates about hinge 84, whereas jaw 454B, comprised in jaw assemblage 454C, is fixed with respect to the hinge and is herein also termed fixed jaw 454B.

Typically each jaw 454 of clamp 450 has a multiplicity of teeth 474 on an inner surface of the jaw, and the teeth of the two jaws are configured to be in opposition to each other. In one embodiment the opposing teeth are configured to intermesh with each other when the jaws are in their closed state.

As is described hereinbelow, clamp 450, once it has been clamped onto a bone of a patient, is registered with the patient. The registration is performed by fluoroscopic imaging of the clamp and the patient, and determining a location of the clamp from the image.

In a disclosed embodiment of the present invention, jaw 454B and jaw assemblage 454C are formed of one or more materials with a common radiopacity, so that there is no part with a different radiopacity. The one or more materials are selected to have a radiopacity different from elements in proximity to clamp 450 when the clamp is used in a surgical procedure. For example, if clamp 450 is used to clamp spinal processes, then the one or more materials have a common radiopacity that is different from the radiopacity of the spinal processes. Forming the common radiopacity to be different from the radiopacity of proximate elements to the clamp facilitates determination of a location of the clamp from a fluoroscopic image.

In the disclosed embodiment, jaw 454B is formed in a predetermined three-dimensional (3D) shape 482B, and the shape is configured so that a location of the jaw can be determined from a fluoroscopic image of the clamp. In one embodiment jaw 454B is formed from aluminum.

Alternatively, jaw 454B may be formed to have multiple different radiopacities, and may, for example, include beads or holes as described above with respect to clamp 350 (FIGS. 8A and 8B).

Turning to FIG. 9, in contrast to marker 66, marker 366 does not comprise radiopaque elements 170, but rather only comprises reflectors 168. Reflectors 168 enable marker 366 to function as a fiduciary for tracking, since the marker still mates with surface 58 (FIGS. 4A, 8A), as is described above for marker 66. However, because of the absence of radiopaque elements 170 in marker 366, the marker does not act as a registration device, and the function of registration is now implemented using the fluoroscopic image of fixed jaw 80B for clamp 350, and of fixed jaw 454B for clamp 450, as described in the following flowchart.

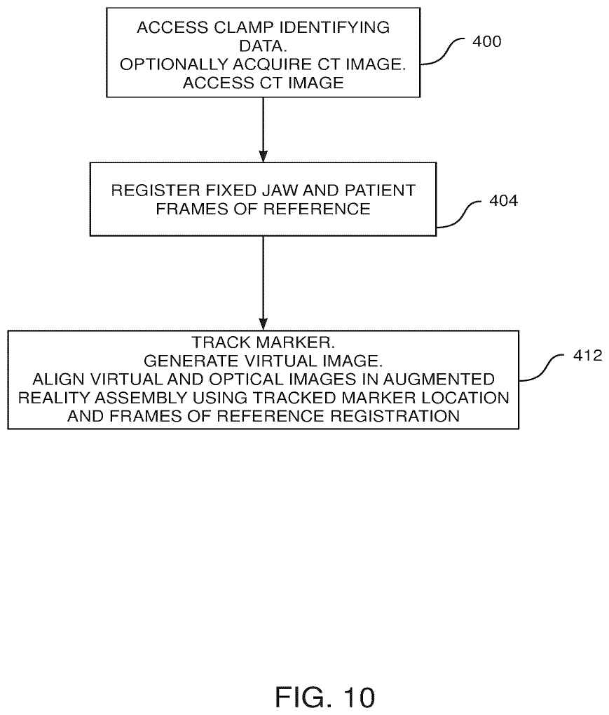

FIG. 10 is a flowchart of steps performed by processor 26 in operation of system 20, using a clamp such as clamp 350 or clamp 450, and marker 366, according to an embodiment of the present invention. Although FIG. 10 is described with respect to the clamps of FIGS. 8A-8E, it may relate to any other clamp having a multi or single radiopacity as herein described. It will also be appreciated that the multi radiopacity feature described herein, (e.g., by including holes or beads as in the clamp of FIGS. 8A and 8B) or the single radiopacity feature (using the clamp shape) may be implemented in other clamps having various shapes and clamping mechanisms, mutatis mutandis. I.e., the clamps of FIGS. 8A-8E are only examples, and all such other clamps referred to above are assumed to be comprised within the scope of the present invention.

Referring to FIG. 10, initially professional 22 inserts clamp 350 or clamp 450 into patient 30 and attaches the clamp to one or more sections of vertebrae of the patient, herein by way of example assumed to be a spinous process of the patient, as is described above for clamp 50. Professional 22 inputs to processor 26 data to be used to identify the location of jaw 80B or jaw 454B. I.e., for jaw 80B locations of selected elements 352 with respect to jaw 80B or predetermined shape 362 of the fixed jaw, and for jaw 454B predetermined shape 480B. It will be understood that such data may be predefined in a production stage of the clamps. Alternatively, the data may be associated with the clamps, and may be input to the processor on selection by the professional of the type of clamp to be used.

With clamp 350 or clamp 450 attached to the patient, in an initial step 400 processor 26 accesses the identifying data referred to above and also accesses a fluoroscopic image, typically a computerized tomography (CT) image, of the clamp and of the patient. Optionally the processor acquires the CT image via an imaging device. It will be understood that the relatively low profile of clamp 350 and clamp 450 facilitates the acquisition of the CT image, since typically the CT fluoroscope acquiring the image has a narrow field of view.

In a registration step 404, processor 26 analyzes the image acquired in step 400 to identify a location of jaw 80B or jaw 454B, by comparing the accessed image with the locations of selected elements 352 or the predetermined shape 362 for jaw 80B, or predetermined shape 482B for jaw 454B, input in step 400. Using the identified location, and images of the spinous process to which clamp 350 or clamp 450 is attached, the processor registers a frame of reference of jaw 80B or jaw 454B with a frame of reference of patient 30. Typically the registration comprises determining a first vector between the location of the jaw and the spinous process to which the clamp is attached. Professional 22 then attaches marker 366 to surface 58 so that when mated with the surface the marker is seated in one of the multiplicity of selectable known positions on the surface described above with reference to FIG. 2B.

In an operational step 412, from the known dimensions of marker 366 and its elements, and because of the known position of the marker with respect to assemblage 80C or 454C, processor 26 determines a second vector, between the location of marker 366 and the clamped spinous process, from the first vector calculated in step 404.

Processor 26 uses camera 72 to acquire an optical image of marker 366 and of patient 30. The processor analyzes the image of marker 366 to track the marker, i.e., to determine its location, comprising its position and orientation, in a frame of reference of system 20, as is described above. Using the second vector generated in the registration and attachment steps described above, the processor determines from the marker location the location of the spinous process which is clamped by clamp 350 or clamp 450, and uses the determined spinous process location to track patient 30.

The processor also may use the optical image acquired by camera 72 to generate a virtual image of the scene viewed by professional 22. The processor registers the virtual image with a frame of reference of patient 30, substantially as described above in virtual image step 208 of the flowchart of FIG. 6, so as to align the virtual image and the acquired optical image.

The processor then projects the registered virtual image in augmented reality assembly 24, for viewing by professional 22. As described above with reference to step 210 of FIG. 7, the professional may introduce a trackable tool 230 into the field of view of camera 72, in which case the processor generates a virtual image of the tool and projects the virtual image in assembly 24 so that it is visible to the professional.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

What is claimed is:

1. A surgical clamp comprising:
a pair of opposing jaws, comprising a movable jaw and a fixed jaw,
wherein each of the opposing jaws comprises a straight proximal region and a distal foot-like region comprising a heel section, a toe section and an arch section connecting the heel section with the toe section, wherein the toe section comprises a narrowed region comprising a flat region at a distal end of the toe section, and
wherein the heel section is connected directly and approximately orthogonally to a distal termination of the straight proximal region so that the distal foot-like region and the straight proximal region reside in a common plane and form one single piece; and
a hinge defining a hinge axis, wherein the opposing jaws reside in respective planes parallel to the hinge axis,
wherein the straight proximal regions of each of the opposing jaws are connected to the hinge to allow the movable jaw to rotate about the hinge between a closed state and an open state of the opposing jaws, and
wherein, in the closed state, the opposing jaws are configured to grip one or more bones of a patient.

2. The clamp of claim 1, wherein each of the opposing jaws comprises a multiplicity of teeth on an inner surface of the respective jaw.

3. The clamp of claim 2, wherein the teeth of the opposing jaws are configured to be in opposition to each other.

4. The clamp of claim 2, wherein the teeth of the opposing jaws are configured to intermesh with each other when the opposing jaws are in their closed state.

5. The clamp of claim 1, further comprising a base and a cover coupled to a proximal end of the straight proximal region of the fixed jaw to form a jaw assemblage that is one single piece.

6. The clamp of claim 5, further comprising a radiopaque marker configured for removable attachment to the cover.

7. The clamp of claim 5, wherein the jaw assemblage is formed of one or more materials with a common radiopacity, and wherein the one or more materials have a radiopacity configured to be different from a radiopacity of elements in proximity to the clamp when the clamp is used in a surgical procedure.

8. The clamp of claim 7, wherein the one or more materials have a radiopacity configured to be different from a radiopacity of spinal elements.

9. The clamp of claim 1, wherein the fixed jaw includes a predetermined part that is radiopaque, so that a location of the fixed jaw is identifiable from a fluoroscopic image of the fixed jaw.

10. The clamp of claim 9, wherein the predetermined part comprises a plurality of holes or beads in predetermined locations along the fixed jaw.

11. The clamp of claim 9, wherein the fixed jaw has a single radiopacity and a predetermined shape, and wherein the predetermined shape is configured so that the fluoroscopic image provides the location of the fixed jaw.

12. A surgical clamp comprising:
a pair of opposing jaws, comprising a movable jaw and a fixed jaw; and
a hinge defining a hinge axis, wherein the opposing jaws reside in respective planes parallel to the hinge axis and terminate at respective proximal regions and distal regions,
wherein the proximal regions are connected to the hinge to allow the movable jaw to rotate about the hinge between a closed state and an open state of the opposing jaws,
wherein, in the closed state, the opposing jaws are configured to grip one or more bones of a patient,
wherein the proximal regions of the opposing jaws are straight or substantially straight,
wherein the distal regions of the opposing jaws comprise a foot-like region,
wherein the foot-like region comprises a heel section, a toe section, and an arch section connecting the heel section with the toe section, and
wherein the toe section comprises a narrowed region comprising a flat region at a distal end of the toe section.

13. The clamp of claim 12, wherein the heel section is connected directly and orthogonally to a distal termination of the proximal region so that the foot-like region and the proximal region form one single piece.

14. The clamp of claim 12, wherein each of the opposing jaws comprises a multiplicity of teeth on an inner surface of the respective jaw.

15. The clamp of claim 12, wherein the fixed jaw is formed to have multiple different radiopacities.

16. The clamp of claim 12, wherein the fixed jaw comprises a plurality of beads or holes in predetermined locations along the fixed jaw.

17. The clamp of claim 12, wherein the fixed jaw is formed in a predetermined three-dimensional shape, and wherein the shape is configured so that a location of the fixed jaw can be determined from a fluoroscopic image of the clamp.
